(19) United States
(12) Patent Application Publication
(10) Pub. No.: US 2018/0074063 A1
(43) Pub. Date: Mar. 15, 2018

(54) Methods and Compositions for Personalized Medicine by Point-of-Care Devices for FSH, LH, HCG and BNP

(71) Applicant: Autotelic LLC, City of Industry, CA (US)

(72) Inventor: Vuong Trieu, Agoura Hills, CA (US)

(73) Assignee: Autotelic LLC, City of Industry, CA (US)

(21) Appl. No.: 15/596,285

(22) Filed: May 16, 2017

Related U.S. Application Data
(63) Continuation of application No. 14/407,715, filed on Dec. 12, 2014, now abandoned.
(60) Provisional application No. 61/659,981, filed on Jun. 15, 2012.
(60) Provisional application No. 61/667,081, filed on Jul. 2, 2012.
(60) Provisional application No. 61/671,717, filed on Jul. 14, 2012.

(51) Int. Cl. G01N 33/574; G01N 33/558; G01N 33/76; G01N 33/68

(52) U.S. Cl. CPC G01N 33/57449; G01N 2800/52; G01N 2333/58; G01N 33/558; G01N 2333/705; G01N 33/689; G01N 33/57492; G01N 33/57488; G01N 2333/59; G01N 33/76

(57) ABSTRACT

The present invention relates to biomarkers, methods, devices, reagent, systems and kits for the detection, diagnosis of ovarian cancer as well as for the monitoring of ovarian cancer progression and for monitoring the progress of various cancer treatmeats including ovarian cancer. The present invention also relates to point-of-care testing (POCT) and methods for determining concentrations of biomarkers in a subject.

### CROSS-REFERENCE TO RELATED APPLICATIONS

This PCT application claims the benefit of priority to U.S. Provisional Applications Nos. 61/659,981 filed Jun. 15, 2012; 61/667,081 filed Jul. 2, 2012 and 61/671717 filed Jul. 14, 2012, which are incorporated by reference in their entirety.

### STATEMENT REGARDING FEDERALLY SPONSORED RESEARCH OR DEVELOPMENT

Not applicable.

### FIELD OF THE INVENTION

The present invention relates to biomarkers, methods, devices, reagent, systems and kits for the detection, diagnosis of ovarian cancer as well as for the monitoring of ovarian cancer progression and for monitoring the progress of various cancer treatments including ovarian cancer. The present invention also relates to point-of-care testing (POCT) and methods for determining concentrations of biomarkers in a subject.

### BACKGROUND OF THE INVENTION

Cancer is a leading cause of death worldwide, accounting for 7.6 million deaths (around 13% of all deaths) in 2008. Prostate cancer is a leading cause of cancer among men. Ovarian cancer is the ninth most common cancer in women and the fifth leading cause of cancer-related deaths in women in the US. One of every 72 women will develop ovarian cancer and one of every 100 will die from this form of cancer. The American Cancer Society estimates that in 2013 22,240 women will be diagnosed with ovarian cancer and about 14,230 will dies from ovarian cancer. About 85% to 90% of ovarian cancers are epithelial ovarian carcinomas.

Treatment options include surgery, chemotherapy, and occasionally radiation therapy. Surgery usually involves the removal of one or both ovaries, fallopian tubes and the uterus. In advanced disease, surgically removing all abdominal metastases enhances the effect of chemotherapy and help improve survival. For women with stage III ovarian cancer in which removal of cancerous tissue has been performed, studies show that chemotherapy administered both intravenously and directly into the peritoneal cavity improves survival.

The identification of tumor markers suitable for early detection and diagnosis of cancer and in particular, ovarian cancer would improve the clinical outcome of patients, especially those presenting vague or no symptoms. Presently no cost effective screening tests have been developed. Ovarian epithelial cancer is more common in individuals with elevated gonadotropin-releasing hormone (GnRH) including follicle-stimulating hormone (FSH) and leutinizing hormone (LH), such as postmenopausal women or women who have received treatment to induce ovulation. Conversely, reduced risk of ovarian cancer is associated with a history of multiple pregnancies, breastfeeding, oral contraceptive use, and estrogen replacement therapy, all of which are related to lower levels of and reduced exposure to FSH and LH. FSH, follicle stimulating hormone, regulates gene expression in ovarian tumors (Chu S, Rushdi S, Zumpe E T, Mamers P, Healy D L, Jobling T, Burger H G, Fuller P J. (2002) FSH-regulated gene expression profiles in ovarian tumours and normal ovaries. Mol Hum Reprod. 8:426-33) and causes neovascularization of ovarian cancers by increasing vascular endothelial growth factor (VEGF) expression through upregulation of survivin (Huang Y, Hua K, Zhou X, Jin H, Chen X, Lu X, Yu Y, Zha X, Feng Y. (2008) Activation of the PI3K/AKT pathway mediates FSH-stimulated VEGF expression in ovarian serous cystadenocarcinoma. Cell Res. 18:780-91).

Currently, cancer antigen 125 (CA-125) is used as a serum biomarker for ovarian cancer. Serum concentrations of CA-125 are elevated in 75-80% of patients with advanced-stage disease and this marker. CA125 is used as a serum tumor marker for monitoring response to chemotherapy, detecting disease recurrence, as well as distinguishing malignant from benign pelvic masses. However, it is presently not an appropriate diagnostic biomarker as the majority of healthy women with high levels of CA-125 do not have cancer.

In addition to a lack of biomarkers for the early detection of cancer including ovarian cancer, the methods of obtaining such biomarkers are also fraught with difficulties. Plasma or serum samples obtained via repetitive venipuncture represent the accepted gold standard for monitoring circulating levels of follicle-stimulating hormone (FSH), luteinizing hormone (LH), estradiol (E2) and progesterone (P) in published reproductive studies. Unfortunately for research subjects, the burden of frequent venipuncture is high; and, for researchers, venipuncture samples require immediate processing and storage facilities with freezers.

Accordingly, there is a need for improved methods of detection and diagnosis of cancer including ovarian cancer as well as methods for monitoring the progress of the disease and monitoring the progress of various treatments for ovarian cancer including point of care or point of use devices capable of quantitating predictive biomarker(s).

### SUMMARY OF THE INVENTION

The present invention relates methods, devices, reagent, systems and kits for the detection of various biomarkers, in particular the measurement of concentrations of biomarkers including gonadotropins.

The present invention relates to biomarkers, methods, devices, reagent, systems and kits for the detection and diagnosis of cancer including ovarian, prostate and testicular cancer.

The present invention relates to biomarkers, methods, devices, reagent, systems and kits for monitoring the progression of ovarian cancer and for monitoring the progress of various cancer treatments.

The present invention also relates to quantitative point-of-care devices and methods for the detection of biomarkers as a diagnostic for various cancers including but not limited to ovarian, prostate and testicular cancer.

In certain embodiments, the present invention provides a method for biomarker monitoring of an individual treated with a drug. The method involves obtaining samples from the individual at suitable time points. The samples may be collected at point-of-care or point-of-use by sampling or self-sampling on point-of-care devices or point of use devices, each capable of quantitating the biomarker , or on matrices suitable for storage of at least two samples prior to quantitation of the drug by a central laboratory. The information obtained may be suitable for guiding dosing of the drug for the individual.

The invention also relates to point-of-care and/or point-of-use devices for quantitation of gonadotropins (hCG, LH, and FSH) as well as BNP which allows for personalized dosing and monitoring for more effective therapies for various cancers including ovarian cancers. In addition, bladder cancers and testicular cancers may be monitored by hCG point-of-care/point-of-use (POC/POU).

Samples may be collected by at point-of-care or point of service, e.g., by self-sampling. Samples may be applied to a lateral flow device for quantitation of the drug, and the results transmitted to the physician or physician's agent for pharmacokinetic analysis. In other embodiments, the samples are collected at point-of-care or point-of-service, e.g., by self-sampling, on a suitable storage matrix, e.g., nitrocellulose, prior to delivery of the samples to a central laboratory for quantitation and analysis.

In certain embodiments, samples collected at various times from the individual through point-of-care or point-of-use by self-sampling may be obtained by a central laboratory. The laboratory then tests the samples to quantitate the biomarker of interest and, based on the results, detection or diagnosis of ovarian cancers may be obtained. The results obtained may also be used to determine the magnitude of the diseases' progression as well as to monitor the efficacy of treatment regimens.

In another aspect it provides a kit for biomarker monitoring of an individual treated with a drug. The kit comprises a plurality of point-of-care device or a point of use device capable of quantitating the drug in one or more samples, or matrices suitable for storage of the samples prior to quantitation by a central laboratory.

### BRIEF DESCRIPTION OF THE FIGURES

FIGS. 1A-C are plots of BNP concentration versus CA125 concentration, FSH concentration versus CA125 concentration and BNP concentration versus FSH concentration, respectively.

FIGS. 2A and B are plots of FSH levels and LH levels determined by POC/POU device in ovarian cancer patients, respectively.

FIGS. 3A is a trace of FSH concentration made from a POC scanner device and 3B is a plot of FSH concentration made from the concentration data measured from a scanner device.

FIG. 4A is a trace of FSH concentration made from a POC smart phone device and 4B is a plot of FSH concentration made from the concentration data measured from a smart phone device.

FIG. 5 is a comparison of traditional assay and expanded range assay for FSH.

FIG. 6A) Scan of traditional lateral flow, B) Scan of lateral flow of one embodiment of the present invention, C) Plot of concentration of FSH of traditional lateral flow and D) Plot of concentration of FSH of lateral flow of one embodiment of the present invention demonstrating the expanded dynamic range for FSH compared to traditional lateral flow.

FIG. 7A) Plot of concentration of hCG with traditional lateral flow and B) Plot of concentration of hCG with lateral flow of one embodiment of the present invention demonstrating the expanded dynamic range for FSH compared to traditional lateral flow and C) Plot of concentration of hCG with lateral flow of one embodiment of the present invention demonstrating the expanded dynamic range for FSH compared to traditional lateral flow.

### DETAILED DESCRIPTION OF THE INVENTION

The present invention relates to biomarkers, methods, devices, reagent, systems and kits for the detection, diagnosis and progression of various cancers as well as to methods by which biomarkers can be measured for determining the efficacy of various cancer treatments. Biomarker data may be obtained from samples collected at point-of-care or point-of-use using for example a lateral flow point of care test. Advantageously, the samples may be obtained by self-sampling. In certain embodiments, the samples may be delivered to a point-of-care device to quantitate the biomarker, and the results thus obtained are reported to the physician or his agent. In one embodiment quantitation of gonadotrophins (hCG, LH, and FSH) as well as BNP can be monitored using point-of-care and point-of-use detection which allows for personalized dosing and monitoring for more effective therapies for various cancers including ovarian cancers. Gonadotropins may be quantitatively detected from various bodily fluids including but not limited to plasma, serum or urine. Point-of-care needs to have an expanded dynamic range of concentration detection. Preferably the assays or test require a single determination with no repeat and no dilution at point-of-care. Most immunoassays have a working range of 2 logs. For example, traditional lateral flow quantitation methods of FSH are limited to a range of only 1-100 IU/L. The methods of the present invention provide for expanding the range of detection for the biomarkers at least one log or at least two logs or at least 3 logs or at least 4 logs or at least 5 logs or at least 6 logs or at least 7 logs or at least 8 logs. For example the assays and tests and methods of the present invention permit an expanded dynamic range of detectable concentrations of FSH between about 1-10,000 IU/L. The expanded dynamic range encompasses all possible concentrations of FSH encountered in blood following administration of clinical dose of FSH or during biomarker testing. Other examples of the uses of the assays, devices, kits and biomarkers of the present invention include applications of hCG quantitation which include but are not limited to: 1) detecting and monitoring pregnancy 2) detecting and managing ectopic pregnancy, 3) determining risk for Down syndrome fetus, 4) predicting preeclampsia, 5) detecting and managing gestational trophoblastic disease, 6) managing testicular germ cell malignancies and 7) monitoring other human malignancies. The challenges for hCG Point-of-Care testing include home use or use in a primary care physician office or emergency room which such that no dilution or manipulation of the sample is important. The sample also should be either blood or urine and the assay must have sufficient dynamic range to accommodate the six logs in range of hCG (0 to >500,000 IU/L). The present invention provide a quantitative point of care assay for hCG that uses urine, plasma, and blood as a sample and has an expanded dynamic range from at least 2-21,000 IU/L instead of traditional method of 2-300 IU/L.

Applications requiring expanded range include but are not limited to: 1) Ectopic Pregnancy: If the hCG is above a certain level 1500 to 2000 mIU/mL, but no pregnancy is seen with ultrasound, an ectopic pregnancy is suspected. If hCG is below 1500 to 2000 mIU/mL and the ultrasound is negative, this may indicate either an ectopic pregnancy or an early intrauterine pregnancy. 2) Pregnancy monitoring: Doubling times for hCG provide a method to determine whether a pregnancy is progressing normally until about six or seven weeks after the last menstrual period. After that point, hCG doubling times begin to slow and ultrasound techniques become the best tool for getting information on how the pregnancy is developing. 3) Emergency department where pregnancy testing is one of the most often ordered tests. 4) Detection and diagnosis of testicular cancer and other germ line cancers.

The assays and tests and methods of the present invention also provide cassettes which can be read using an optical reader with 2D barcode capability, and provide that the data can be printed out or stored on the reader for uploading onto a database including but not limited to a clinic, doctor's office or hospital database. The reader utilizes confocal optics with a low distance-to-target ratio. The reflectometric measurement is converted to activity units, using an established calibration curve embedded in the 2D barcode. The cassettes are made such that the samples within are stable for at least 12 or at least 24 or at least 48 or at least 72 hours and thus can be shipped to a central lab or doctor's office for quantitation if the patient does not have access to the reader.

The readers utilized in the present invention include but are not limited to confocal optical readers or cell/smart phone readers. A reflectometric optical reader, which utilizes confocal optics with a low distance-to-target ratio, may be sued with the methods, kits and assays of the present inventions. Calculations may be performed in the background using information embedded on the 2D-bar code specific to each lot of cassettes. Alternatively a cell phone reader which provides quantitation at a point-of-care without sufficient resources can be used to capture images of the cassettes and transmit the images over the Internet to a facility such as a centralized processing facility where the FSH or other biomarker values can be received in real time.

The present invention provides rapid and quantitative point-of-care testing for gonadotropins including FSH for field deployment directly at home, and provides for cassettes which can be read directly by the patient or can be shipped to the central lab/doctor's office for reading. As TDM, the test should allow for more effective dosing of the patients and thereby improving effectiveness of hormone manipulation therapy or cancer treatment. The tests are also patient-centric, inviting better compliance and patient participation in personalizing his/her treatment. The simplicity of the assays would allow for their deployment in underdeveloped regions lacking access to central laboratories with specialized and expensive equipment. The expanded range eliminates the need for dilution of the samples to bring them within working range of the traditional assay.

Alternatively, the samples are collected using a matrix or vessel suitable for collection and storage of the samples until receipt and analysis by a central laboratory. Examples of matrices or vessels suitable for collection and storage of the samples include, but are not limited to commercially available biological sampling filter paper systems such as Whatman 3 MM, GF/CM30, GF/QA30, S&S 903, GB002, GB003, or GB004. Several categories of blotting materials for blood specimen collection are available, e.g., S&S 903 cellulose (wood or cotton derived) filter paper and Whatman glass fiber filter paper. The blood spot is placed in one or more designated areas of the filter paper, allowed to dry, and then mailed along with a test request form to the central laboratory. This method of collection has the advantage of obviating the need for collection of samples at a doctor's office or clinic. Thus, multiple samples may be conveniently collected by the patient over a period of 0 to 72 hours at considerable savings of cost and time. This has the advantages of increased efficiency and reduced delays in transmitting results of the analysis to the treating physician, who may use the information to adjust treatment as necessary, and contact the patient to convey the new treatment regimen. In one aspect, one or more biomarkers are provided for use either alone or in various combinations to diagnose ovarian cancer, permit differential diagnosis of pelvic masses as benign or malignant, monitor ovarian cancer progression or monitor ovarian cancer recurrence.

Any of the biomarkers described herein may be used in a variety of clinical indications for ovarian cancer, including any of the following: detection of ovarian cancer, characterizing ovarian cancer (e.g. determining ovarian cancer type, sub-type or stage), such as by determining whether a pelvic mass is benign or malignant; determining ovarian cancer prognosis; monitoring ovarian cancer progression or remission; monitoring for ovarian cancer recurrence; monitoring metastasis; treatment selection (e.g. pre- or post-operative chemotherapy selection; monitoring response to a therapeutic agent or other treatment, combining biomarker testing with additional biomedical information.

As an example of the manner in which the biomarkers described herein may be used to diagnose ovarian cancer, differential expression of one or more of the biomarkers described herein in an individual who is not known to have ovarian cancer may indicate that the individual has ovarian cancer thereby enabling detection of ovarian cancer at an early stage of the disease when treatment is most effective. Increased expression of the biomarker from “normal” during the course of ovarian cancer may be indicative of ovarian cancer progression whereas a decrease in the expression as compared with normal expression may indicate that the individual is in remission or is being successfully treated. Increases in the degree of biomarker expression as compared to “normal” may indicate cancer progression or ineffectiveness of ovarian cancer treatment. Additionally an increase or decrease in the differential expression of one or more of the biomarkers after an individual has apparently been cured of ovarian cancer may be indicative or ovarian cancer recurrence. In this case, cancer treatment may be resumed or current treatment may be augmented or supplemented as need be. In addition, a differential change in the level of biomarker might also be indicative of an individual's response to a particular therapeutic agent. Differential expression refers to expression of a biomarker that is activated to a higher or lower level in a subject suffering from a specific disease, relative to its expression in a normal or control subject or a subject who does not have the specific disease. Differential expression includes both quantitative and qualitative differences in expression among normal and diseased cells or among cells which have undergone different disease events or different treatments.

The biomarkers of the present invention include CA-125, follicle stimulating hormone (FSH) and brain natriuretic peptide (BNP) which may be used individually or in combination. FSH is released by the anterior pituitary gland and, stimulates production of eggs and estradiol during the first half of the menstrual cycle in women. Brain natriuretic peptide is a 32 aa, ˜3 kDa peptide encoded by the human NPPB gene and plays a role in the modulation of diuresis, vasorelaxation and secretion of renin and aldosterone.

A plurality of biomarkers in a sample may increase the sensitivity and/or specificity of a particular test.

Biomarkers may be differentially expressed at any level, but the biomarker or biomarkers are generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 150%, by at least 200%, by at least 500% or by at least 1000%. The level of expression of the biomarker(s) of the present invention may be between about 10% higher to about 250% higher or about 10% higher to about 150% higher or about 10% higher to about 125% higher or about 10% higher to about 100% higher or from about 10% higher to about 90% higher or from about 10% higher to about 80% higher or from about 10% higher to about 70% higher or from about 10% higher to about 60% higher or from about 10% higher to about 50% higher or from about 10% higher to about 40% higher or from about 10% higher to about 30% higher or about 10% higher to about 20% higher or about 20% higher to about 125% higher or about 20% higher to about 100% higher or from about 20% higher to about 90% higher or from about 20% higher to about 80% higher or from about 20% higher to about 70% higher or from about 20% higher to about 60% higher or from about 20% higher to about 50% higher or from about 20% higher to about 40% higher or from about 50% higher to about 250% or from about 50% higher to about 125% higher or from about 50% higher to about 100% higher than normal or than that of a control.

A biomarker is preferably differentially present at a level that is statistically significant (e.g. a p-value less that 0.05 and/or a q-value of less than 0.10 as determined by either Welch's T-test or Wilcoxon's rank-sum test). Alternatively, the biomarkers demonstrate a correlation with the presence of ovarian cancer or particular stages of ovarian cancer. The range of correlations is between negative 1 (−1), a perfect negative correlation, and positive 1 (+1), a perfect positive correlation. Zero (0) would mean no correlation. A substantial positive correlation refers to a biomarker having a correlation between +0.25 and +1.0 with a disease or clinical measurement while a substantial negative correlation refers to a biomarker having a correlation between −0.25 and −1.0 with a disease or clinical measurement. A significant positive correlation refers to a biomarker having a correlation between +0.25 and +1.0 with a disease or clinical measurement while a significant negative correlation refers to a biomarker having a correlation between −0.25 and −1.0 with a disease or clinical measurement.

In some cases it will be desirable to establish normal or baseline values (or ranges) for biomarker expression levels. Normal levels can be determined for any particular population, subpopulation or group according to standard methods known to those of skill in the art. Generally baseline (normal) levels of biomarkers are determined by quantifying the amount of biomarker in biological samples (e.g. fluids, cells or tissues) obtained from normal (healthy) individuals. Application of standard statistical methods permits determination of baseline levels of expression as well as deviations from such baseline levels.

A biomarker value for the biomarkers of the present invention can be detected by using any of a variety of known analytical methods. Biomarker detection may be facilitated by the use of a capture agent which is one or more molecules which can specifically bind the biomarker. The capture agent, in solution or immobilized on a solid support, may be exposed to the biomarker and binding may be detected in a variety of ways including but not limited to fluorescence, chemiluminescence, dyes, and other optically detectable means. Immunoassay methods are based on the binding of an antibody to its corresponding analyte and can detect the analyte in a sample depending on the specific assay format.

According to the American Cancer Society ovarian cancer can be staged according to the AJCC/TNM System. This describes the extent of the primary tumor (T), the absence or presence of metastasis to nearby lymph nodes (N), and the absence or presence of distant metastasis (M). T categories for ovarian cancer include: T1: The cancer is confined to one or both ovaries; T2: The cancer is in one or both ovaries and is extending into pelvic tissues and T3: The cancer is in one or both ovaries and has spread to peritoneum. N categories indicate if the cancer has spread to regional (nearby) lymph nodes. Nx: No description of lymph node involvement is possible because information is incomplete. N0: No lymph node involvement. N1: Cancer cells are found in the lymph nodes close to tumor. Once a patient's T, N, and M categories have been determined, this information is combined in a process called stage grouping to determine the stage, expressed in Roman numerals from stage I (the least advanced stage) to stage IV (the most advanced stage). Stage 1: The cancer is still contained within the ovary (or ovaries). It has not spread outside the ovary.. Stage 2: The cancer is in one or both ovaries and has spread to other organs (such as the uterus, fallopian tubes, bladder, the sigmoid colon, or the rectum) within the pelvis. It has not spread to lymph nodes, the peritoneum, or distant sites. Stage 3: The cancer is in one or both ovaries, and one or both of the following are present: (1) cancer has spread beyond the pelvis to the lining of the abdomen; (2) cancer has spread to lymph nodes. Stage 4: This is the most advanced stage of ovarian cancer. In this stage the cancer has spread to the inside of the liver, the lungs, or other organs located outside the peritoneal cavity. Finding ovarian cancer cells in the fluid around the lungs is also evidence of stage IV disease.

Non-limiting examples of suitable devices or methods of testing drugs include lateral flow devices for the determination of the concentration of an analyte in a sample comprising providing a lateral flow strip for use in measuring the analyte. Examples of analytes that may be tested include therapeutic drugs, drug metabolites, and hormones. Application of the sample to the lateral flow strip causes a fraction of the analyte in the sample to bind to a component of the lateral flow strip such that a detectable signal proportional to the concentration of the analyte in the sample is produced.

Alternatively, the quantitation may be conducted on samples submitted by individuals to a laboratory by any suitable assay, including, but not limited to, those currently known to the art, such as ELISA, liquid chromatography-mass spectrometry (LC-MS), thin layer chromatography (TLC), high-performance liquid chromatography (HPLC), and mass spectrometry (MS) or other traditional assays for drug monitoring at central lab have been well illustrated. The samples could be whole blood collected following a finger prick on a suitable matrix and stored as a dry blood spot that is shipped or otherwise delivered to a laboratory for testing. Sampling can be performed with capillary and/or device designed to deliver precise and small amount of blood to the dried blood spot card- card punch variability replaced pipette variability.

### EXAMPLES

### Example 1

### CA125 Monitoring for Personalized Dosing

Serum samples collected at time of diagnosis of ovarian cancer (N=136) were tested for CA-125 and the data was evaluated using JMP9 statistical analysis software. Serum samples (N=136) were collected at the time of patients were diagnosed with ovarian cancer. ELISA method was performed to detect the CA125 level in patient serum

TABLE 1

| Stage | N | Mean | Std Dev. | Lower 95% | Upper 95% |
|---|---|---|---|---|---|
| I | 20 | 209.57 | 307.71 | 65.56 | 353.6 |
| II | 22 | 350.02 | 418.14 | 164.63 | 535.4 |
| III | 79 | 820.58 | 1225.66 | 546.05 | 1095.1 |
| IV | 14 | 1897.78 | 1598.98 | 974.56 | 2821.0 |

CA125 level was higher for stage IV than other stages [I (N=20; 210 +308 U/mL), II (N=22; 350±418 U/mL), III (N=79; 821±1226 U/mL), IV (N=14; 1898±1599 U/mL)], p<0.0001, <0.0001, 0.0009 vs. I, II, and III, respectively).

TABLE 2

| T | N | Mean | Std Dev. | Lower 95% | Upper 95% |
|---|---|---|---|---|---|
| T1 | 20 | 209.57 | 307.71 | 65.56 | 353.6 |
| T2 | 22 | 350.02 | 418.14 | 164.63 | 535.4 |
| T3 | 94 | 985.37 | 1329.05 | 713.15 | 1257.6 |

CA125 level was higher for T3 than T2 and T1 [T1 (N=20; 210±308 U/mL), T2 (N=22; 350±418 U/mL), T3 (N=94; 985±1329 U/mL)], p=0.0061, and 0.0190 vs. T1 and T2, respectively.

TABLE 3

| N | N | Mean | Std Dev. | Lower 95% | Upper 95% |
|---|---|---|---|---|---|
| N1 | 10 | 1757.19 | 1812.32 | 460.74 | 3053.6 |

CA125 level was higher for N1 than N0 [N0 (N=125; 694±1077 U/mL) and N1 (N=10; 1757±1812 U/mL)], p=0.005.

TABLE 4

| M | N | Mean | Std Dev. | Lower 95% | Upper 95% |
|---|---|---|---|---|---|
| M0 | 121 | 634.03 | 1043.92 | 446.13 | 821.9 |
| M1 | 15 | 1853.26 | 1550.43 | 994.66 | 2711.9 |

CA125 level was higher for M1 than M0 [M0 (N=121; 634±1044 U/mL) and M1 (N=15; 1853±1550 U/mL)], p<0.0001. CA125 level was higher for clear cell histological subtype than other subtypes [clear cell (N=13; 1471±1307 U/mL), endometrioid (N=23; 574±442 U/mL), mucinous (N=16; 255±272 U/mL), and serous (N=83; 808±1337 U/mL)], p=0.005, p=0.0259, p=0.055 vs. mucinous, endometrioid, and serous subtypes, respectively.

TABLE 5

| | | | | Lower | Upper |
|---|---|---|---|---|---|
| Subtype | N | Mean | Std Dev. | 95% | 95% |
| Clear cell | 13 | 1471.22 | 1307.29 | 681.24 | 2261.2 |
| Endometrioid | 23 | 573.76 | 442.20 | 382.54 | 765.0 |
| mucinous | 16 | 255.09 | 272.10 | 110.09 | 400.1 |
| serous | 83 | 808.29 | 1337.26 | 516.29 | 1103.3 |

CA-125 level was higher for clear cell histological subtype than other subtypes [clear cell (N=13; 1471±1307 U/mL), endometrioid (N=23; 574±442 U/mL), mucinous (N=16; 255±272 U/mL), and serous (N=83; 808±1337 U/mL)], p=0.005, p=0.0259, p=0.055 vs. mucinous, endometrioid, and serous subtypes, respectively. CA125 level was higher for stage IV than other stages [I (N=20; 210±308 U/mL), II (N=22; 350±418 U/mL), III (N=79; 821±1226 U/mL), IV (N=14; 1898±1599 U/mL)], p<0.0001, <0.0001, 0.0009 vs. I, II, and III, respectively). CA125 level was higher for T3 than T2 and T1 [T1 (N=20; 210±308 U/mL), T2 (N=22; 350±418 U/mL), T3 (N=94; 985±1329 U/mL)], p=0.0061, and 0.0190 vs. T1 and T2, respectively. CA125 level was higher for N1 than N0 [N0 (N=125; 694±1077 U/mL) and N1 (N=10; 1757±1812 U/mL)], p=0.005. CA125 level was higher for M1 than M0 [M0 (N=121; 634±1044 U/mL) and M1 (N=15; 1853±1550 U/mL)], p<0.0001. The data support the use of CA-125 levels for monitoring the disease status in ovarian cancer patients.

### Example 2

### Measurement of LH. FSH and BNP Levels

Serum samples collected at time of diagnosis of ovarian cancer were tested using rapid and quantitative point-of-care (POC) devices for blood biomarkers (LH, FSH, and BNP) and the data was evaluated using JMP9 statistical analysis software. Quantitative lateral flow assays for FSH and LH were performed according to Lam Hwang, Chao Hsiao, Kouros Motamed, Vuong Trieu (2012) Rapid and Quantitative Lateral Flow Point-of-Care Therapeutic Drug Monitoring (TDM) Assays for LH and FSH. American Association for Cancer Research (AACR) Annual Meeting, Mar. 31-Apr. 4, 2012. Quantitative BNP assay was from Humasis (Korea). FSH range=5-10,000 IU/L; LH range=1-1,700 IU/L; BNP range=25˜800 pg/mL.

TABLE 6

| BNP (pg/ml) | | | | | |
|---|---|---|---|---|---|
| Quartiles | 10% | 25% | median | 75% | 95% |
| Normal | 0 | 0 | 0 | 84.1 | 177.3 |
| Ovarian Cancer | 0 | 0 | 40.0 | 74.8 | 127.3 |

p = 0.02, chi-square

TABLE 7

| LH (pg/ml) | | | | | |
|---|---|---|---|---|---|
| Quartiles | 10% | 25% | median | 75% | 95% |
| Normal | 4.83 | 6.00 | 16.65 | 62.53 | 83.72 |
| Ovarian Cancer | 4.50 | 12.60 | 17.40 | 40.00 | 61.20 |

p = 0.9817 Wilcoxon statistics

TABLE 8

| BNP (pg/ml) | | | | | |
|---|---|---|---|---|---|
| Quartiles | 10% | 25% | median | 75% | 95% |
| Normal | 3.79 | 7.75 | 13.40 | 99.68 | 828.84 |
| Ovarian Cancer | 15.10 | 74.30 | 151.60 | 418.60 | 825.00 |

p = 0.0102 Wilcoxon statistics

In the serous adenocarcinoma group, FSH level was higher (median=151.6 mU/ml) vs. normal controls (median of 13.4 mU/ml, p=0.01, Wilcoxon). Moreover, incidence of BNP>25 pg/ml was higher for patients (14 of 19, 74%) vs. normal controls (3 of 10, 30%, p=0.02, Chi-square). FSH progressively increased from normal controls, to normotensive patients, to hypertensive patients with median FSH values of 13.4, 79.3, and 232.2, respectively. The same was not observed for BNP. FSH increase in hypertensive patients was not accompanied by increase in BNP. However, incidence of BNP>25 pg/ml was higher for patients (14 of 19, 74%) vs. normal controls (3 of 10, 30%, p=0.02, Chi-square. No differences were observed for LH. These data suggest the BNP and FSH hormones play a role(s) in ovarian cancer.

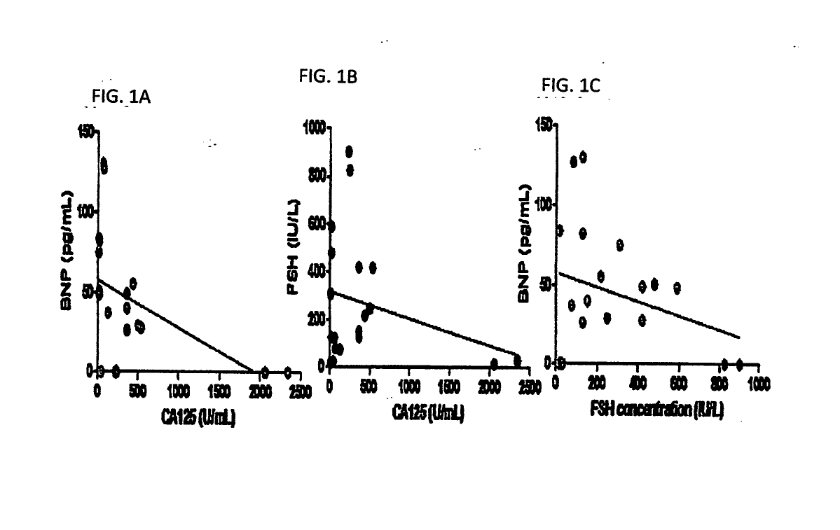

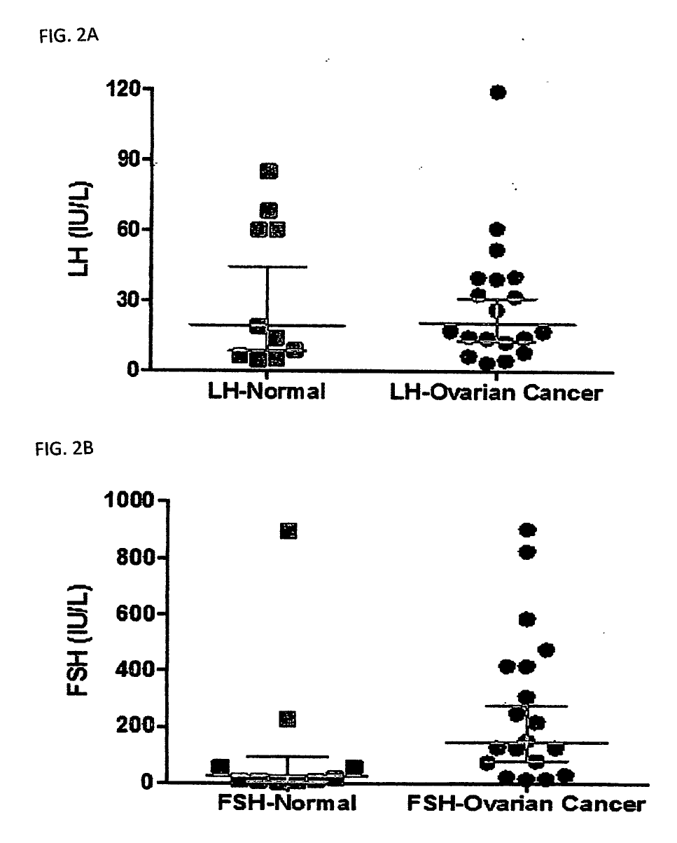

There were no correlations between CA125 and FSH or BNP suggesting that they are independent biomarkers (FIG. 1). FSH and BNP exhibited increased incidence/level among cancer patients versus age matched normal. FSH is probably acting as regulator of cancer cell expression as well as induction of angiogenesis. Elevated levels of FSH are shown in FIG. 2. The role of BNP is unknown at the moment. The data shown in this example demonstrated the use of POC/POU device for monitoring the relevant blood biomarkers (FSH, LH, hCG, and BNP). It is surprising that BNP was associated with ovarian cancer.

### Example 3

### Measurement of LH, FSH and BNP Levels

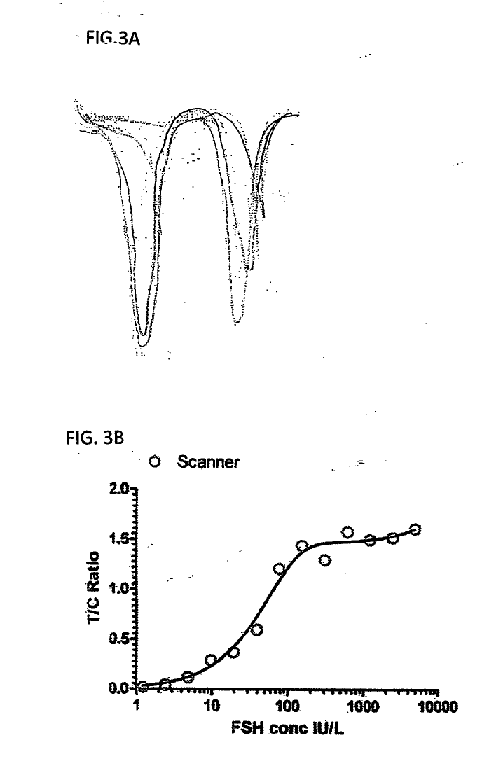

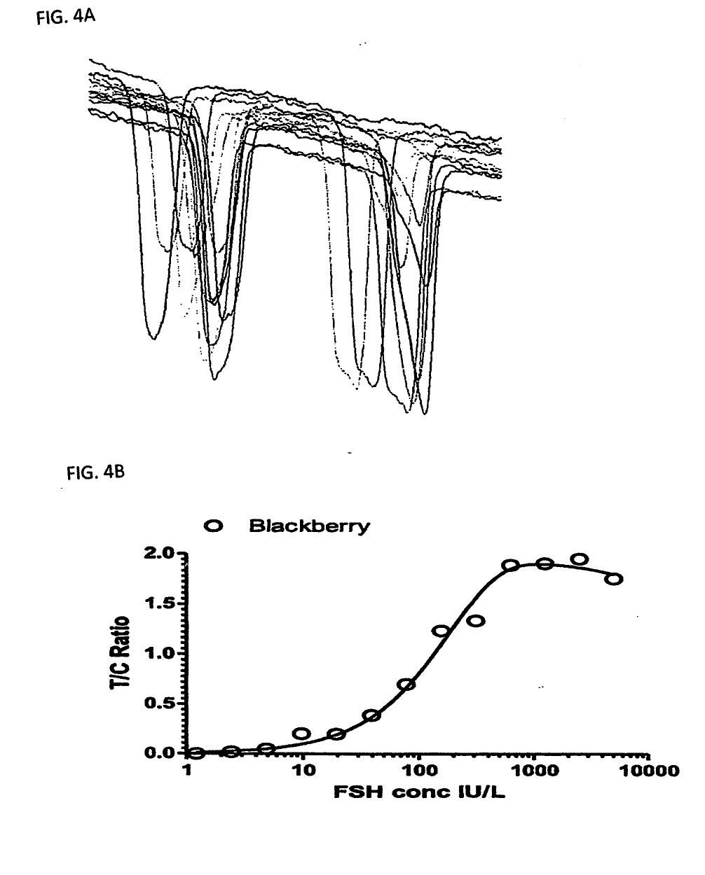

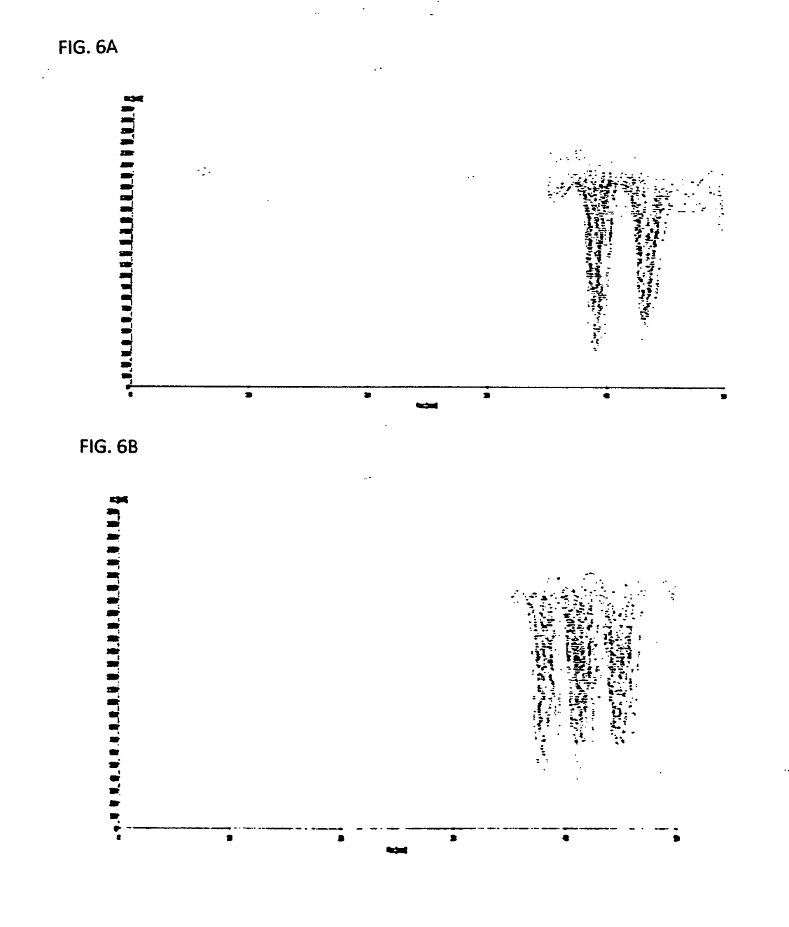

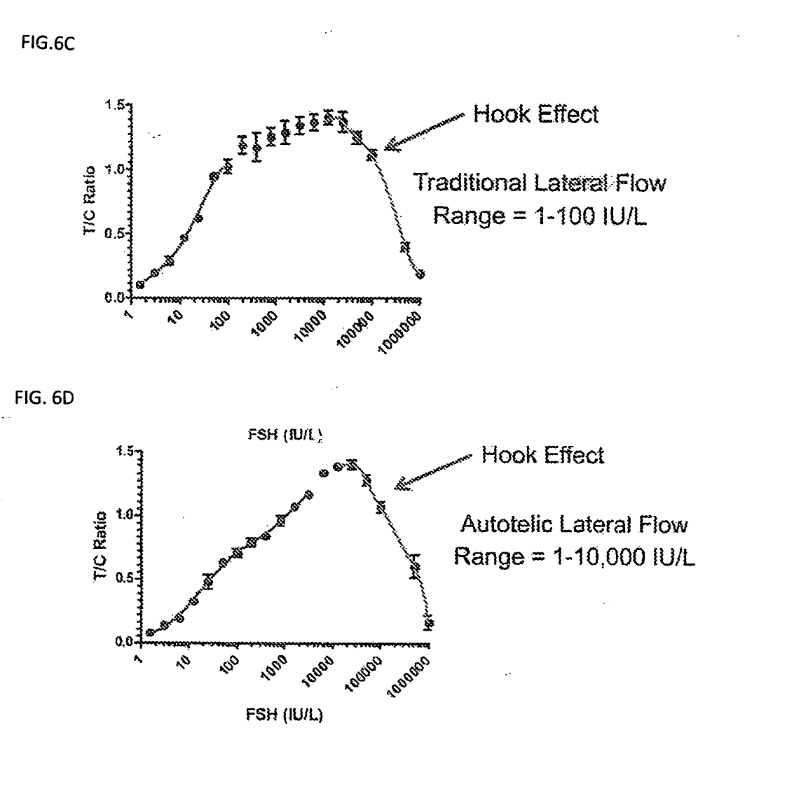

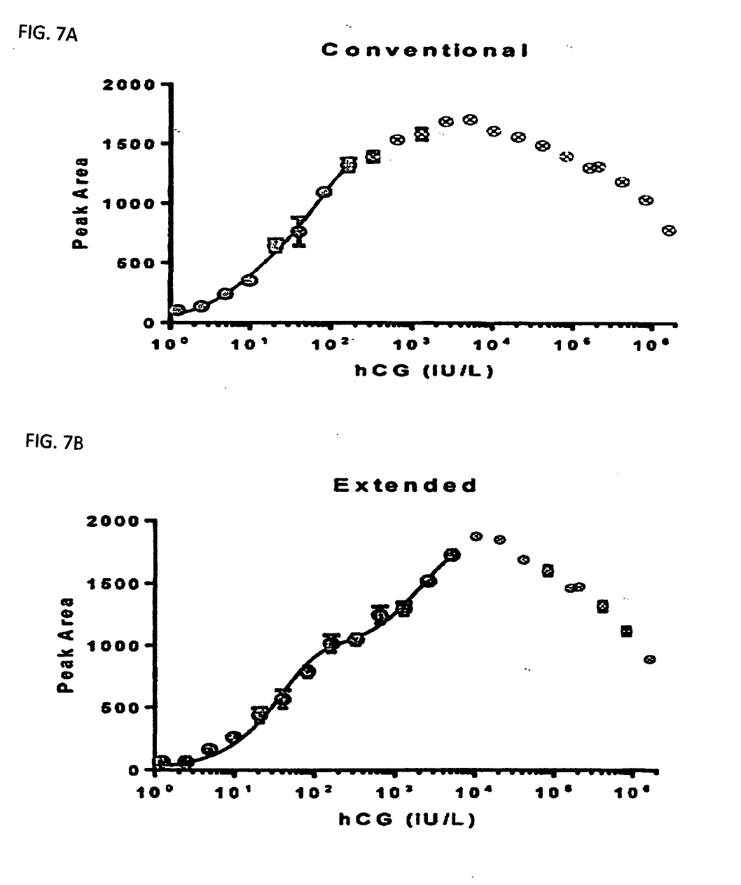

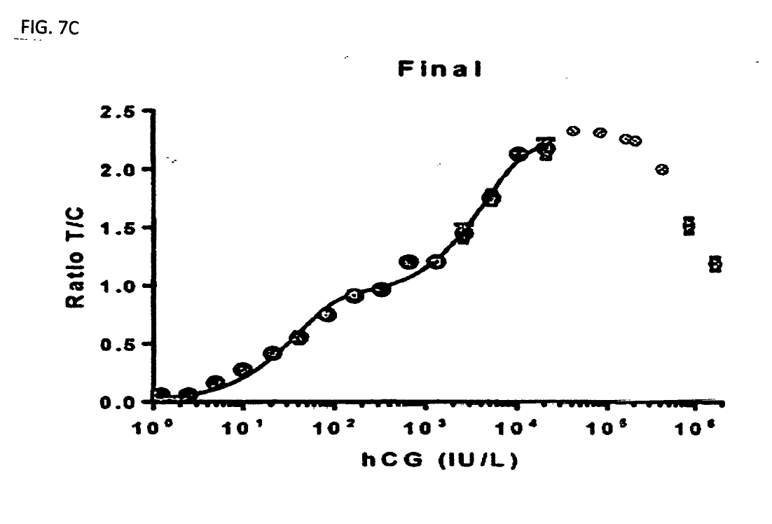

Rapid and quantitative lateral flow point of care test (POCT) for FSH was developed using proprietary method to achieve increased dynamic range suitable for TDM. Serum samples collected at time of diagnosis of ovarian cancer and from normal individuals were tested for FSH using the POCT assay. Clinical data were analyzed using JMP9 statistical analysis software. The data shown in FIGS. 3 and 4, demonstrate that confocal optical readers or cell phone cameras such as the blackberry can be used to generate quantifiable image for quantitation using either an on board image analyzer or an image analyzer program at central lab.

Where ranges are given herein, the endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While this invention has been particularly shown and described with references to example embodiments thereof, it will be understood by those skilled in the art that the various changes in form and details may be made therein without departing from the scope of the invention encompassed by the appended claims.

Further advantages of the present immunological compositions and adjuvants of the present invention can be achieved by those skilled in the art based upon the embodiments described herein and are thus specifically within the scope of the present invention.

1. A method of monitoring the efficacy of an ovarian cancer treatment in a patient comprising
determining the level of one or more biomarker(s) selected from the group consisting of hCG, FSH, BNP, LH and CA-125 and combinations thereof in the patient at a first point in time after diagnosis of ovarian cancer in a patient, wherein the level of biomarker(s) is determined using a lateral flow device with an expanded dynamic range;
treating the patient with an ovarian cancer treatment;
determining the level of the biomarker(s) in the patient at a first point in time;
comparing the level(s) of the biomarker(s) in the patient at the first point in time with the level(s) of the biomarker(s) in the patient at the second point in time to determine the differential expression of the biomarker(s).

2-3. (canceled)

4. The method of claim 1, wherein the level of biomarker in the patient is differentially expressed by at least 10%.

5. The method of claim 1, wherein the level of biomarker in the patient is differentially expressed by at least 50%.

6. (canceled)

7. The method of claim 1, wherein the biomarker is FSH.

8. The method of claim 1, wherein the biomarker is hCG.

9. The method of claim 1, wherein the biomarker is LH.

10. The method of claim 1, wherein the biomarker is BNP.

11-31. (canceled)

32. The method of claim 1, wherein the biomarker is CA-125

33. The method of claim 1, wherein the determination of the level of biomarker in the patient is performed by quantitative point of care devices.

34. The method of claim 1 further comprising altering the ovarian cancer treatment of the patient based upon the differential expression of the biomarker(s) from the first point in time to the second point in time.

35. . The method of claim 1 wherein the level of two or more biomarkers are determined.

36. . The method of claim 1 wherein the expanded dynamic range is least four logs.

37. . The method of claim 1 wherein the expanded dynamic range is least five logs.

38. The method of claim 1 wherein the expanded dynamic range is least six logs.

39. The method of claim 1 wherein the expanded dynamic range is least seven logs.

40. The method of claim 1 wherein the biomarker is hCG and range of detection is from about 0 to 500,000 2-21000 IU/L.

41. The method of claim 1 wherein the expanded dynamic range lateral flow device can detect FSH in the range from 1 to 10,000 IU/L.

42. The method of claim 1 wherein the expanded dynamic range lateral flow device can detect BNP in the range from 25 to 800 pg/ml.

43. The method of claim 1 wherein the difference between the level of biomarker in the patient and the normal level is statistically significant.
